(19) United States
(12) Patent Application Publication
(10) Pub. No.: US 2010/0080842 A1
(43) Pub. Date: Apr. 1, 2010

(54) TRANSDERMAL EXTENDED-DELIVERY DONEPEZIL COMPOSITIONS AND METHODS FOR USING THE SAME

(76) Inventors: Jianye Wen, Palo Alto, CA (US); Yoshiko Katori, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/551,231

(22) Filed: Aug. 31, 2009

Related U.S. Application Data
(60) Provisional application No. 61/101,412, filed on Sep. 30, 2008.

(51) Int. Cl. A61K 9/70; A61K 31/445

(52) U.S. Cl. 424/448; 514/319; 424/400; 424/449

(57) ABSTRACT

A transdermal extended-delivery donepezil active agent composition is provided. Aspects of the compositions of the invention include a donepezil active agent layer that is formulated to provide for multi-day delivery of a therapeutically effective amount of a donepezil active agent to a subject when the composition is topically applied to the subject. Also provided are methods of using the formulations, e.g., for administering a donepezil active agent to a subject, and kits containing the formulations.

### CROSS-REFERENCE TO RELATED APPLICATIONS

Pursuant to 35 U.S.C. §119 (e), this application claims priority to the filing dates of: U.S. Provisional Patent Application Ser. No. 61/101,412 filed on Sep. 30, 2008; the disclosure of which application is herein incorporated by reference.

### INTRODUCTION

Alzheimer's disease is a degenerative brain disease that causes dementia, a progressive decline in cognitive function beyond what might be expected from normal aging. Short-term memory loss is the most common symptom, and later symptoms include confusion, anger, mood swings, language breakdown, long-term memory loss, and the general withdrawal of the subject as his or her senses decline. Alzheimer's disease has no current cure, however its symptoms can be treated with active agents, such as acetylcholinesterase inhibitors (e.g., donepezil, galantamine, rivastigimine, tacrine, etc.) and N-methyl D-aspartate (NMDA) receptor antagonists (e.g., memantine).

Donepezil, known chemically as (±)-2,3-dihydro-5,6-dimethoxy-2-[[1-(phenylmethyl)-4-piperidinyl]methyl]-1H-inden-1-one, is a reversible acetylcholinesterase inhibitor that is used to treat the symptoms of Alzheimer's disease. Typically, donepezil is provided as donepezil hydrochloride in tablet form for oral administration (e.g., Aricept®, Pfizer, Inc., New York).

Transdermal active agent formulations, also known as transdermal patches or skin patches, are adhesive patches containing an active agent that are placed on the skin to deliver the active agent through the skin. Transdermal patches deliver the active agent by percutaneous absorption, which is the absorption of substances through unbroken skin. After a transdermal patch is applied to the skin, the active agent contained in the patch passes through, or permeates the skin and can reach its site of action through a systemic blood flow. Alternatively, the transdermal patch may be placed on the desired treatment site such that the medication contained in the patch is delivered topically.

### SUMMARY

A transdermal extended-delivery donepezil active agent composition is provided. Aspects of the compositions of the invention include a donepezil active agent layer that is formulated to provide for multi-day delivery of a therapeutically effective amount of a donepezil active agent to a subject when the composition is topically applied to the subject. Also provided are methods of using the formulations, e.g., for administering a donepezil active agent to a subject, and kits containing the formulations.

### BRIEF DESCRIPTION OF THE FIGURES

FIG. 1 shows a cross sectional view of an embodiment of the transdermal active agent formulation described herein.

FIGS. 2 to 4 graphically represent results reported in the Experimental Section, below.

### DETAILED DESCRIPTION

A transdermal extended-delivery donepezil active agent composition is provided. Aspects of the compositions of the invention include a donepezil active agent layer that is formulated to provide for multi-day delivery of a therapeutically effective amount of a donepezil active agent to a subject when the composition is topically applied to the subject. Also provided are methods of using the formulations, e.g., for administering donepezil active agent to a subject, and kits containing the formulations.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be constructed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various embodiments of the invention, aspects of the transdermal donepezil compositions are reviewed first in greater detail, followed by a detailed description of methods of using the compositions and a review of, kits that include the transdermal formulations.

### Transdermal Anti-Dementia Active Agent Formulations

As summarized above, transdermal donepezil compositions are provided. The compositions of the invention include a donepezil active agent layer, wherein the donepezil active agent layer is formulated to provide for multi-day delivery of a therapeutically effective amount of a donepezil active agent to a subject when said composition is topically applied to said subject. By multi-day delivery is meant that the layer is formulated to provide a therapeutically effective amount to a subject when the composition is applied to a skin site of a subject for a period of time that is 2 days or longer, e.g., 3 days or longer, such as 5 days or longer, including 7 days or longer, such as 10 days or longer. By therapeutically effective amount is meant that the compositions when applied to a skin site of a subject during its intended time of application, e.g., within 7 days of application, provides for a systemic amount of donepezil that provides a desired therapeutic activity. In some embodiments, the compositions provide delivery of a target dosage of donepezil that is 5 mg/day or greater over a one week period (i.e., 7 days or 168 hours), including 10 mg/day or greater over one week, such as 15 mg/day or greater over one week. The active agent compositions of embodiments of the invention are formulated to provide for high skin permeation rates, e.g., as determined using the skin permeation assay reported in the Experimental Section, below. In certain embodiments, skin permeation rates of 1.5 μg/cm²/hr or greater, such as 2.5 μg/cm²/hr or greater, including 3.5 μg/cm²/hr or greater are provided by the compositions.

The size (i.e., area) of the transdermal compositions may vary. In certain embodiments, the size of the composition is chosen in view of the desired transdermal flux rate of the active agent and the target dosage. For example, if the transdermal flux is 3.4 μg/cm²/hr and the target dosage is 5 mg/day, then the transdermal composition is chosen to have an area of about 43 cm². Or for example, if the transdermal flux is 3.4 μg/cm²/hr and the target dosage is 10 mg/day, then the transdermal patch is chosen have an area of about 87 cm². In certain embodiments, the compositions have dimensions chosen to cover an area of skin when applied to a skin site that ranges from 10 to 200, such as 20 to 150, including 40 to 140 cm².

The donepezil active agent layer of the compositions may vary in thickness. In some instances, the thickness of the active agent layer ranges from 25 to 250, such as 50 to 200, including 100 to 150 micrometers in thickness.

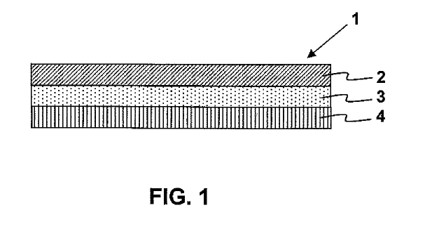

In some embodiments, the compositions of the invention include a donepezil active agent layer, a backing layer and release liner. For example, FIG. 1 a composition 1 according to an embodiment of the invention, where the composition 1 includes a backing layer 2, a donepezil active agent layer 3, and a release liner 4. Each of these layers is now described in greater detail.

### Donepezil Active Agent Layer

The donepezil active agent layer of compositions of the invention includes a donepezil active agent. By donepezil active agent is meant donepezil freebase or a salt thereof, e.g., donepezil hydrochloride. Donepezil freebase has the empirical formula of C₂₄H₂₉NO₃ and the IUPAC name (±)-2,3-dihydro-5,6-dimethoxy-2-[[1-(phenylmethyl)-4-piperidinyl]methyl]-1H-inden-1-one. Donepezil has the following chemical structure:

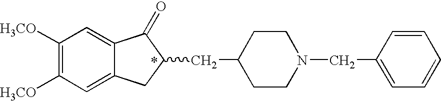

Salts of donepezil may include the hydrochloride salt, and the like. Donepezil hydrochloride salt, or donepezil-HCl, has the empirical formula of C₂₄H₂₉NO₃.HCl and the IUPAC name (±)-2,3-dihydro-5,6-dimethoxy-2-[[1-(phenylmethyl)-4-piperidinyl]methyl]-1H-inden-1-one hydrochloride. Donepezil-HCl has the following chemical structure:

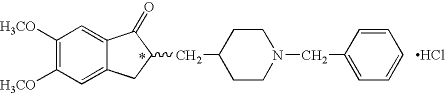

The amount of donepezil active agent present in the donepezil active agent layer is sufficient to provide for the desired extended delivery of donepezil to a subject when applied to a skin site of a subject. In certain embodiments, the donepezil active agent layer includes a donepezil active agent in an amount ranging from 10% to 35% (w/w), such as 15 to 30% (w/w), including 20 to 25% (w/w). In certain embodiments, the donepezil active agent layer is free of solid and un-dissolved donepezil active agent. This means that the donepezil active agent layer does not include crystalline or other solid forms of the donepezil active agent, or donepezil active agent that is not present in the composition.

In addition to the donepezil active agent, embodiments of the donepezil active agent layer include a percutaneous absorption enhancer. Percutaneous absorption enhancers employed in embodiments of the compositions facilitate the absorption of the donepezil active agent by the skin of the subject. Accordingly, the percutaneous absorption enhancer may also be referred to as a percutaneous permeation enhancer because it may facilitate not only the percutaneous absorption of the active agent, but also the percutaneous permeation of the active agent through the skin of the subject.

Of interest as percutaneous absorption enhancers are polyoxyethers of alcohols, such as but not limited to polyoxyethers of aliphatic alcohols, including saturated or unsaturated higher alcohols, e.g., having 8 to 22 carbon atoms, such as oleyl alcohol and lauryl alcohol. In certain embodiments, the percutaneous absorption enhancer is described by the formula:

CₘH₂ₘ₊₁(OCH₂CH₂)ₙOH

wherein:

m is an integer ranging from 8 to 22, such as 8 to 18; and

n is an integer ranging from 2 to 25, such as 2 to 23.

Specific percutaneous absorption enhancers of interest include laureth-4 and laureth-23, as well as combinations thereof.

In some cases, the donepezil active agent layer contains the percutaneous absorption enhancer in an amount ranging from 2% to 25% (w/w), such as from 10% to 25% (w/w), and including from 15% to 25% (w/w), where 15% (w/w) is present in certain embodiments.

In certain embodiments, the transdermal donepezil composition of the invention is provided in an adhesive format, such as an adhesive tape or an adhesive patch. In certain of these embodiments, the donepezil active agent layer is an adhesive layer, such that when the composition is applied to a skin surface the composition is adhered to a skin surface by the adhesion of the active agent layer to the skin surface.

In certain of these embodiments, the donepezil active agent layer includes a pressure-sensitive adhesive. The terms “pressure-sensitive adhesive”, “self adhesive”, and “self-stick adhesive” mean an adhesive that forms a bond when pressure is applied to adhere the adhesive with a surface. In some instances, the adhesive is one in which no solvent, water, or heat is needed to activate the adhesive.

Pressure sensitive adhesives of interest include, but are not limited to acrylate copolymers. Acrylate copolymers of interest include copolymers of various monomers which may be “soft” monomers, “hard” monomers, and optionally “functional” monomers. Also of interest are blends including such copolymers. The acrylate copolymers can be composed of a copolymer including bipolymer (i.e., made with two monomers), a terpolymer (i.e., made with three monomers), or a tetrapolymer (i.e., made with four monomers), or copolymers made from even greater numbers of monomers. The acrylate copolymers can include cross-linked and non-cross-linked polymers. The polymers can be cross-linked by known methods to provide the desired polymers.

Monomers from which the acrylate copolymers are produced include at least two or more exemplary components selected from the group including acrylic acids, alkyl acrylates, methacrylates, copolymerizable secondary monomers or monomers with functional groups. Monomers (“soft” and “hard” monomers) of interest include, but are not limited to, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers are described in Satas, “Acrylic Adhesives,” Handbook of Pressure-Sensitive Adhesive Technology, 2nd ed., pp. 396-456 (D. Satas, ed.), Van Nostrand Reinhold, New York (1989).

Of interest are acrylate copolymers that include polar functional monomeric residues. Of specific interest are monomeric residues that provide for —COOH functional groups. Useful carboxylic acid monomers to provide the —COON functional group may contain from about 3 to about 6 carbon atoms and include, among others, acrylic acid, methacrylic acid, itaconic acid, and the like. Acrylic acid, methacrylic acid and mixtures thereof are employed in certain embodiments acids. The functional monomer(s) are present in certain embodiments of the copolymers in an amount of 2 wt % or more, such as between 3-10 wt %.

In some embodiments, the adhesive may have a composition that is, or is substantially the same as, the composition of DuroTak® 87-2852 (National Adhesives, Bridgewater, N.J.). The term “substantially the same” as used herein refers to a composition that is an acrylate-vinyl acetate copolymer in an organic solvent solution and provides for the functionality as described herein. In some embodiments, the acrylic pressure-sensitive adhesive is DuroTak® 87-2852.

In some embodiments, the adhesive may have a composition that is, or is substantially the same as, the composition of DuroTak® 87-2054 (National Adhesives, Bridgewater, N.J.). The term “substantially the same” as used herein refers to a composition that is an acrylate-vinyl acetate copolymer in an organic solvent solution and provides for the functionality as described herein. In some embodiments, the acrylic pressure-sensitive adhesive is DuroTak® 87-2054.

In some embodiments, the adhesive may have a composition that is, or is substantially the same as, the composition of DuroTak® 87-2196 (National Adhesives, Bridgewater, N.J.). The term “substantially the same” as used herein refers to a composition that is an acrylate-vinyl acetate copolymer in an organic solvent solution and provides for the functionality as described herein. In some embodiments, the acrylic pressure-sensitive adhesive is DuroTak® 87-2196.

Other examples of polyacrylate-based adhesives of interest are as follows, identified as product numbers, manufactured by National Starch (DURO-TAK® is a trademark of National Starch adhesives): 87-200A, 87-2353, 87-2100, 87-2051, 87-2052, 87-2194, 87-2677, 87-201A, 87-2979, and 87-2074.

### Backing Layer

As summarized above, compositions of the invention may include a backing layer. The backing may be flexible to an extent that it can be brought into close contact with a skin surface. In certain embodiments, the backing is such that it does not absorb the active agent, and does not allow the active agent to be released from the backing side. The backing may include, but is not limited to, non-woven fabrics, fabrics, films (including sheets), porous bodies, foamed bodies, paper, composite materials obtained by laminating a film on a non-woven fabric or fabric, and combinations thereof.

Non-woven fabric may include, but is not limited to the following: polyolefin resins such as polyethylene and polypropylene; polyester resins such as polyethylene terephthalate, polybutylene terephthalate and polyethylene naphthalate; and besides rayon, polyamide, polyester ether), polyurethane, polyacrylic resins, polyvinyl alcohol, styrene-isoprene-styrene copolymers, and styrene-ethylene-propylene-styrene copolymers; and combinations thereof. Fabric may include, but is not limited to cotton, rayon, polyacrylic resins, polyester resins, polyvinyl alcohol, and combinations thereof.

The film may include, but is not limited to the following: polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof.

The paper may include, but is not limited to impregnated paper, coated paper, wood free paper, Kraft paper, Japanese paper, glassine paper, synthetic paper, and combinations thereof. Composite materials may include, but are not limited to composite materials obtained by laminating the above-described film on the above-described non-woven fabric or fabric.

### Release Liner

In some embodiments, a release liner is provided on the donepezil active agent layer, and specifically on a surface of the active agent layer that is distal (i.e. opposite) from the backing layer, if present. The release liner facilitates the protection of the active agent layer. The release liner may be prepared by treating one side of polyethylene-coated wood free paper, polyolefin-coated glassine paper, a polyethylene terephthalate (polyester) film, a polypropylene film, or the like with a silicone treatment.

### Adhesive Overlay

Optionally, an adhesive overlay can be used to increase the adhesion of the composition when applied to the skin. Adhesive overlays can include a layer of adhesive present on a backing material, such as a porous, non-porous, occlusive, or breathable backing material. The dimensions of the adhesive overlay are chosen to provide the desired functionality, where in some instances the dimensions are chose such that the adhesive overlay, when applied over the active agent formulation, extends some distance beyond one or more of the sides of the active agent formulation. In some instances, the area of the adhesive overlay exceeds the area of the active agent formulation by 5% or more, such as by 10% or more, including by 20% or more. During use, the adhesive overlay can be applied by the patients, by the care givers, or can be integrated in the kits.

### Methods

Methods for administering a donepezil active agent to a subject are also provided. In certain embodiments, the methods include applying to a skin site of the subject a transdermal donepezil active agent composition of the invention, e.g., as described in detail above, and maintaining the composition at the skin site of the subject for a period of time sufficient to deliver the donepezil active agent to the subject. The transdermal active agent composition may be applied to the skin of the subject, for example at a skin site, a keratinized skin site, etc. The transdermal active agent composition may be applied to a skin surface of a desired skin site such that the composition is adhered to the skin surface by the adhesion of the active agent layer to the skin surface.

The transdermal active agent composition may be applied to a skin site for an amount of time sufficient to deliver the donepezil active agent to the subject. In some cases, the transdermal active agent composition may be applied to the skin site for an amount of time sufficient to deliver an effective amount of the donepezil active agent to the subject. The term “effective amount” means a dosage sufficient to provide the desired result. For example, an effective amount may be an amount of the donepezil active agent present in the composition that is sufficient such that, when applied to a skin site in accordance with the methods described herein, the subject's symptoms associated with Alzheimer's disease and/or dementia are alleviated at least to some measurable extent (e.g., as determined by using any convenient art accepted assay), if not completely diminished.

In some embodiments, the transdermal active agent composition may be applied to the skin site for an amount of time sufficient to deliver a target dose of the active agent to the subject over a period of time. The target dose that is delivered may be one that provides for a systemic level of active agent that is sufficient to provide the desired activity with respect to the target disease, e.g., Alzheimer's. For example, the target dose of the active agent may be 5 mg/day or greater, including 10 mg/day or greater, such as 15 mg/day or greater. In some cases, the transdermal active agent composition may be applied to the skin site for an amount of time ranging from 1 day to 14 days, such as 3 days to 10 days, including 7 days to 10 days. In certain cases, the transdermal active agent composition may be applied to the skin site for 7 days (i.e., one week).

After the transdermal active agent composition has been applied to the skin site for the desired amount of time (i.e., an amount of time sufficient to deliver a target dose of the active agent to the subject over a period of time), the composition may be removed from the skin site. A new transdermal composition may be applied at the same or at a different skin site. The new transdermal composition may be applied to a different skin site to reduce the possible occurrence of skin irritation and/or skin sensitization at the prior site of application.

In certain embodiments, the methods described herein may include a diagnostic step. Individuals may be diagnosed as being in need of the subject methods using any convenient protocol, and are generally known to be in need of the subject methods, e.g., they are suffering from a target disease condition or have been determined to be at risk for suffering from a target disease condition, prior to practicing the subject methods.

Diagnosis or assessment of Alzheimer's disease and dementia is well-established in the art. Assessment may be performed based on, but not limited to the following: patient history; collateral history from relatives; diagnostic tests, such as clinical observation of behavior; mental status testing of cognitive functions including but not limited to memory, language, perceptual skills, attention, constructive abilities, orientation, problem solving and functional abilities; physical examinations; neurological examinations; brain imaging, such as but not limited to computed tomography (CT), magnetic resonance imaging (MRI), positron emission tomography (PET), and single photon emission computed tomography (SPECT); and the like.

### Utility

The transdermal active agent compositions find use in any application where a subject would benefit from being administered an antidementia active agent, such as but not limited to donepezil. In certain embodiments, the compositions are employed in the treatment of a condition. By “treatment” is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition.

In general, administration of donepezil according to the subject methods can be used to treat diseases or conditions including, but not limited to Alzheimer's disease, dementia, and the like. The transdermal active agent composition may be used for administering donepezil to a subject. In these cases, the method includes applying a transdermal active agent composition, as described herein, to a skin surface of a subject. The method further includes maintaining the active agent composition on the skin of the subject for a period of time sufficient to deliver the active agent to the subject. Subjects of interest include humans.

In certain embodiments, the transdermal active agent composition is provided as an adhesive patch and is applied to the skin surface, whereby the active agent in the composition can be administered by percutaneous permeation through the skin.

When the transdermal active agent composition is applied to a skin surface, the active agent permeates the skin in contact with the patch to reach the site of action through a systemic blood flow.

### Kits

Kits for use in practicing the methods described herein are also provided. In certain embodiments, the kits include a transdermal donepezil active agent composition, e.g., as described above. In certain embodiments, the kits include an adhesive overlay as described above. In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

The following examples are offered by way of illustration and not by way of limitation. Specifically, the following examples are of specific embodiments for carrying out the present invention. The examples are for illustrative purposes only, and are not intended to limit the scope of the present invention in any way.

### EXAMPLES

### I. Materials and Methods

### A. Preparation of Active Agent Reservoir Layer

Formulations were prepared by mixing stock solutions of each of the mixture components in organic solvents (typically 30-60 wt % solid content in ethyl acetate, methanol and/or ethanol), followed by a mixing process. Once a homogeneous mixture was formed, the solution was cast on a release liner (siliconized polyester sheet of 2-3 mils) and dried at 65°-80° C. for 10-90 minutes. The adhesive films were laminated to a PET backing.

### B. Transdermal Flux Tests

Human cadaver skin was used and epidermal layers (stratum corneum and epidermis) were separated from the full-thickness skin as skin membrane. Samples were die-cut with an arch punch to a final diameter of about 2.0 cm². The release liner was removed and the system was placed on top of the epidermis/stratum corneum with the drug adhesive layer facing the stratum corneum. Gentle pressure was applied to effect good contact between the adhesive layer and stratum corneum. The donor and receptor sides of the Franz cell were clamped together and the receptor solution containing a phosphate buffer at pH 6.5 was added to the Franz cell. The cells were kept at 33° C. for the duration of the experiment. Samples of the receptor solution were taken at regular intervals and the active agent concentration was measured by HPLC. The removed receptor solution was replaced with fresh solution to maintain the sink conditions. The flux was calculated from the slope of cumulative amounts of the drug in the receiver compartment versus time plot.

### C. Specific Examples

### C.1 Effect of Enhancer Loading

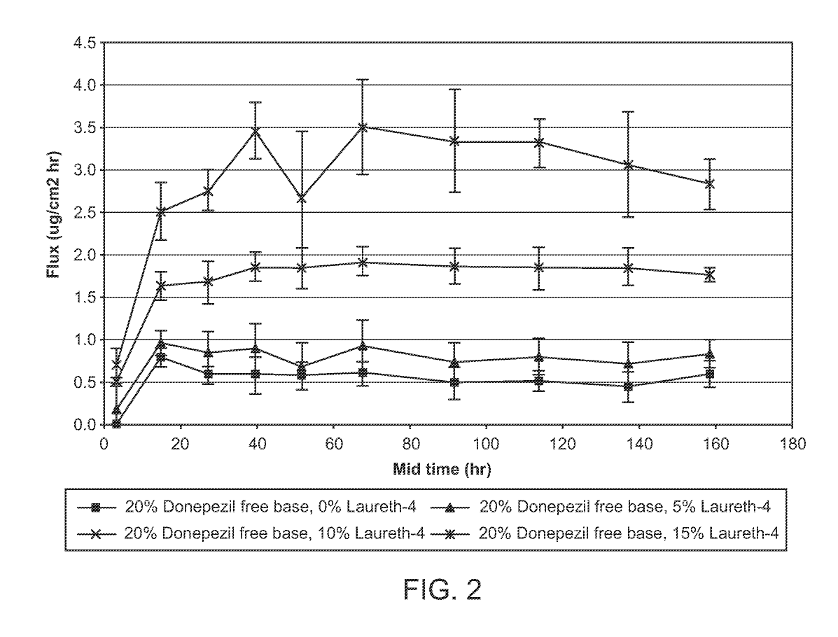

Using the general method described previously, a series transdermal systems containing 0 to 15% laureth-4 were prepared with details shown in following table. The steady state flux through human cadaver skin was estimated to increase from 0.5 μg/cm².hr to 3.3 μg/cm².hr when laureth-4 loading was increased from 0 to 15%. The results are provided in Table 1, below as well as graphically presented in FIG. 2.

TABLE 1

| | Formulation | | | Steady state flux, |
|---|---|---|---|---|
| Sample | Adhesive | Done-pezil | Laureth-4 | μg/cm² · hr |
| 1 | Duro-tak 87-2852 | 20% | 0% | 0.5 |
| 2 | Duro-tak 87-2852 | 20% | 5% | 0.8 |
| 3 | Duro-tak 87-2852 | 20% | 10% | 1.9 |
| 4 | Duro-tak 87-2852 | 20% | 15% | 3.3 |

### C.2 Effect of Enhancer Structure

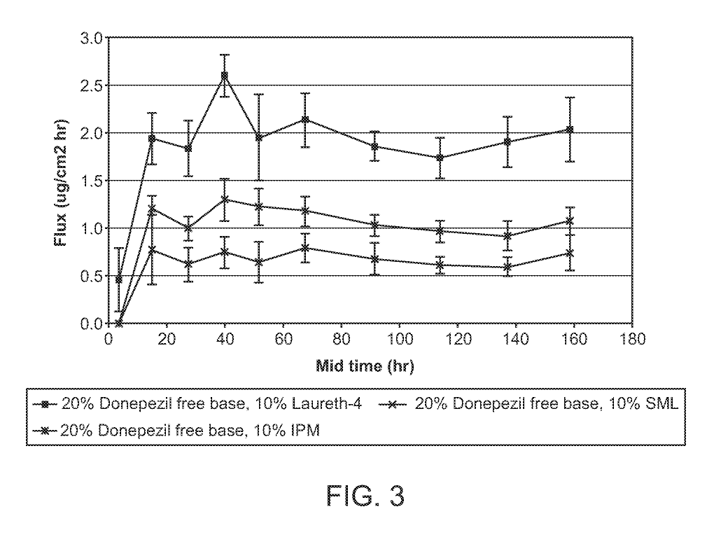

Using the general method described previously, a series of transdermal systems containing different enhancers were prepared. The results are provided in Table 2 below and graphically FIG. 3.

TABLE 2

| | Formulation | | | Steady state flux, |
|---|---|---|---|---|
| Sample | Adhesive | Donepezil | Enhancer | μg/cm² · hr |
| 1 | Duro-tak 87- | 20% | 10% laureth4 | 1.9 |
| | 2852 | | | |
| 2 | Duro-tak 87- | 20% | 10% SML | 0.7 |
| | 2852 | | | |
| 3 | Duro-tak 87- | 20% | 10% IPM | 1.0 |
| | 2852 | | | |
| 4 | Duro-tak 87- | 20% | None | 0.5 |
| | 2852 | | | |

### C.3 Flux in Different Adhesives

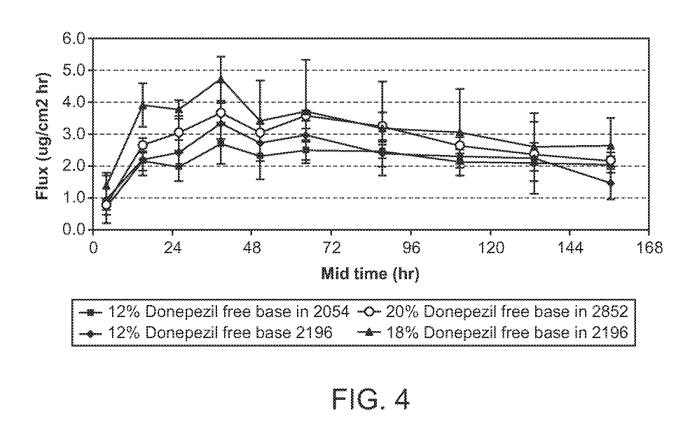

Using the general method described previously, transdermal systems using different adhesives were prepared. Results are provided in Table 3 below and in FIG. 4.

TABLE 3

| | Formulation | | | Steady state flux, |
|---|---|---|---|---|
| Sample | Adhesive | Donepezil | Enhancer | μg/cm² · hr |
| 1 | Duro-tak 87- | 12% | 10% | 2.5 |
| | 2054 | | laureth4 | |
| 2 | Duro-tak 87- | 20% | 10% | 3.0 |
| | 2852 | | laureth4 | |
| 3 | Duro-tak 87- | 12% | 10% | 2.7 |
| | 2196 | | laureth4 | |
| 4 | Duro-tak 87- | 18% | 10% | 3.4 |
| | 2196 | | laureth4 | |

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An extended delivery transdermal donepezil active agent composition, said composition comprising:
a donepezil active agent layer, wherein said donepezil active agent layer is formulated to provide for multi-day delivery of a therapeutically effective amount of a donepezil active agent to a subject when said composition is topically applied to said subject.

2. The extended delivery transdermal donepezil active agent composition according to claim 1, wherein said donepezil active agent layer comprises:
(a) said donepezil active agent;
(b) a percutaneous absorption enhancer; and
(c) a pressure sensitive adhesive.

3. The extended delivery transdermal donepezil active agent composition according to claim 1, wherein said donepezil active agent layer has a thickness ranging from 50 to 200 μm.

4. The extended delivery transdermal donepezil active agent composition according to claim 1, wherein said composition is formulated to provide for seven-day or longer delivery of a therapeutically effective amount of a donepezil active agent to a subject when said composition is topically applied to said subject

5. The extended delivery transdermal donepezil active agent composition according to claim 1, wherein said donepezil active agent layer comprises a donepezil active agent in an amount ranging from 10% to 25% (w/w).

6. The extended delivery transdermal donepezil active agent composition according to claim 5, wherein said donepezil active agent is donepezil freebase.

7. The extended delivery transdermal donepezil active agent composition according to claim 1, wherein said donepezil active agent layer is free of solid and un-dissolved donepezil active agent.

8. The extended delivery transdermal donepezil active agent composition according to claim 2, wherein said percutaneous absorption enhancer is a polyoxyether alcohol.

9. The extended delivery transdermal donepezil active agent composition according to claim 8, wherein said polyoxyether alcohol is described by the formula:
CₘH₂ₘ₊₁(OCH₂CH₂)ₙOH
wherein:
m is an integer ranging from 8 to 18; and
n is an integer ranging from 2 to 23.

10. The extended delivery transdermal donepezil active agent composition according to claim 9, wherein said percutaneous absorption enhancer is
laureth-4.

11. The extended delivery transdermal donepezil active agent composition according to claim 9, wherein said percutaneous absorption enhancer is
laureth-23.

12. The extended delivery transdermal donepezil active agent composition according to claim 2, wherein said pressure sensitive adhesive comprises an acrylic polymer.

13. The extended delivery transdermal donepezil active agent composition according to claim 11, wherein said acrylic polymer is an acrylate copolymer.

14. The extended delivery transdermal donepezil active agent composition according to claim 13, wherein said acrylic polymer comprises carboxylic acid functionalities.

15. The extended delivery transdermal donepezil active agent composition according to claim 14, wherein said pressure sensitive adhesive is substantially the same as or is DURO-TAK 87-2852® pressure sensitive adhesive.

16. The extended delivery transdermal donepezil active agent composition according to claim 1, wherein said composition is formulated to provide a skin permeation rate of said donepezil active agent of 1.5 μg/cm²/hr or greater.

17. The extended delivery transdermal donepezil active agent composition according to claim 16, wherein said composition is formulated to provide a skin permeation rate of said donepezil active agent of 2.5 μg/cm²/hr or greater.

18. The extended delivery transdermal donepezil active agent composition according to claim 1, wherein said composition further comprises a backing layer.

19. The extended delivery transdermal donepezil active agent composition according to claim 1, wherein said composition further comprises a release liner.

20-24. (canceled)

25. A method for administering a donepezil active agent to a subject, said method comprising:
(a) applying to a skin site of said subject an extended delivery transdermal donepezil active agent composition, said composition comprising a donepezil active agent layer, wherein said donepezil active agent layer is formulated to provide for multi-day delivery of a therapeutically effective amount of a donepezil active agent to a subject when said composition is topically applied to said subject; and
(b) maintaining said composition at said skin site of said subject for a period of time sufficient to deliver said active agent to said subject.

26. The method according to claim 25, wherein said donepezil active agent layer comprises:
(a) said donepezil active agent;
(b) a percutaneous absorption enhancer; and
(c) a pressure sensitive adhesive.

27. The method according to claim 26, wherein said donepezil active agent layer has a thickness ranging from 50 to 200 μm.

28. The method according to claim 25, wherein said composition is formulated to provide for seven-day or longer delivery of a therapeutically effective amount of a donepezil active agent to a subject when said composition is topically applied to said subject and said method comprises maintaining said composition at said skin site for seven days or longer.

29. The method according to claim 26, wherein said donepezil active agent layer comprises a donepezil active agent in an amount ranging from 10% to 25% (w/w).

30. The method according to claim 29, wherein said donepezil active agent is donepezil freebase.

31. The method according to claim 30, wherein said donepezil active agent layer is free of solid and un-dissolved donepezil active agent.

32-43. (canceled)

44. A kit comprising:
an extended delivery transdermal donepezil active agent composition, wherein said composition comprises a donepezil active agent layer that is formulated to provide for multi-day delivery of a therapeutically effective amount of a donepezil active agent to a subject when said composition is topically applied to said subject; and
an adhesive overlay.

45-62. (canceled)
